(19) United States
(12) Patent Application Publication
(10) Pub. No.: US 2013/0273158 A1
(43) Pub. Date: Oct. 17, 2013

(54) EXTENDED RELEASE COMPOSITIONS OF QUETIAPINE

(71) Applicants: Bandi Parthasaradhi Reddy, Hyderabad (IN); Podili Khadgapathi, Hyderabad (IN); Kappala Ramesh, Hyderabad (IN)

(72) Inventors: Bandi Parthasaradhi Reddy, Hyderabad (IN); Podili Khadgapathi, Hyderabad (IN); Kappala Ramesh, Hyderabad (IN)

(73) Assignee: Hetero Research Foundation, Hyderabad (IN)

(21) Appl. No.: 13/446,013

(22) Filed: Apr. 13, 2012

(51) Int. Cl. A61K 31/554; A61K 9/36; A61P 25/18; A61K 9/00

(52) U.S. Cl. 424/472; 514/211.13; 424/400

(57) ABSTRACT

Described herein are pharmaceutical compositions of quetiapine, more particularly extended release compositions of quetiapine or its pharmaceutically acceptable salts comprising carboxymethyl ethyl cellulose, a non-gelling, hydrophobic release controlling polymer, and processes for preparing the same.

### FIELD OF THE DISCLOSURE

The technical field of the present disclosure relates to pharmaceutical compositions, more particularly extended release compositions comprising quetiapine.

### BACKGROUND OF THE DISCLOSURE

Quetiapine fumarate is a psychotropic agent belonging to a chemical class of dibenzothiazepine derivatives, designated chemically as 2-[2-(4-dibenzo [b,f][1,4]thiazepin-1 1-yl-1-piperazinyl)ethoxy]-ethanol fumarate (2:1)(salt). Its molecular formula is C₄₂H₅₀N₆O₄S₂.C₄H₄O₄ having a molecular weight of 883.11. The structural formula is:

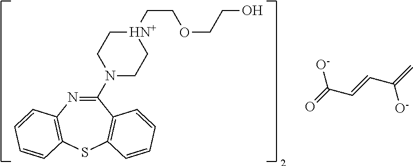

Quetiapine is marketed as immediate release as well as extended release tablets in United States under the trade name Seroquel® and Seroquel XR®, respectively, by AstraZeneca.

U.S. Pat. No. 4,879,288 discloses 11-[4-[2-(2-hydroxyethoxy)ethyl]-1-piperazinyl]dibenzo [b, f] [1, 4] thiazepine as an antipsychotic drug of dibenzothiazepine class suitable for treatment of various psychotic disorders.

U.S. Pat. No. 5,948,437 discloses sustained release formulations of quetiapine using gelling agents such as hydroxypropyl methylcellulose and its derivatives that create a gel structure after contact with water.

U.S. Pat. No. 4,547,571 describes process for the preparation of carboxymethyl ethyl cellulose (CMEC) polymer.

WO 2004012699 discloses modified release dosage forms prepared by using dual retard technique comprising micro matrix particles containing quetiapine and hydrophobic release controlling agents, which are coated with hydrophobic release controlling agents.

WO 2005041935 discloses matrix composition comprising quetiapine and a wax material.

WO 2007086079 discloses sustained release compositions of quetiapine comprising a channelizer and rate controlling polymer.

WO 2008060228 discloses extended release compositions comprising quetiapine, hydroxypropyl methyl cellulose and sodium citrate dihydrate.

WO 2009113051 discloses sustained release compositions containing quetiapine and one or more non-gelling and/or waxy excipients.

WO 2010001413 discloses sustained release dosage forms comprising quetiapine or its pharmaceutically acceptable salts and one or more non-gellable release controlling polymers.

WO 2010028794 discloses a matrix formulation in the form of a retard tablet comprising quetiapine, at least one matrix-forming, water-insoluble, non-swellable auxiliary agent, and at least one water-soluble binding agent.

Extended release formulations for oral administration are beneficial for a number of reasons. For example, they enable the patient to ingest the formulation less frequently, which may lead to improved patient compliance with the dosing regimen. They may also result in fewer side effects. These formulations may also provide a longer plateau concentration of the drug in the blood. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Swellable matrix systems have disadvantages in case of high doses of soluble active pharmaceutical ingredients as it may be difficult to control the release profile of the active pharmaceutical ingredient. This is due to an increasing diffusion rate caused by continuous dissolution of the drug from the matrix, which process accelerates the penetration of water into the matrix, resulting in faster dissolution and release of the drug. The use of waxes also requires melting and cooling of the wax to produce the dosage form, and making the manufacture more time consuming. These drawbacks can be overcome by the present disclosure.

### SUMMARY

It has been surprisingly found that pharmaceutical compositions comprising carboxymethyl ethyl cellulose (CMEC), a non-gelling, hydrophobic release controlling polymer exhibited excellent release characteristics that were also found to be comparable with the marketed formulation of Seroquel XR®.

The present disclosure relates to an extended release pharmaceutical compositions comprising quetiapine or its pharmaceutically acceptable salts, polymorphs, solvates, hydrates thereof, carboxymethyl ethyl cellulose (CMEC), a non-gelling, hydrophobic release controlling polymer, and one or more pharmaceutically acceptable excipients.

The present disclosure also relates to extended release tablet compositions comprising quetiapine, carboxymethyl ethyl cellulose, a non-gelling, hydrophobic release controlling polymer, and one or more pharmaceutically acceptable excipients.

In one embodiment, carboxymethyl ethyl cellulose and quetiapine are present in the core and/or in a coating of the tablet that is disposed on the core.

In an embodiment, the present disclosure provides an extended release composition comprising quetiapine and one or more excipients selected from release controlling polymers, diluents, disintegrants, binders, lubricants, glidants and plasticizers; characterized in that said composition comprise carboxymethyl ethyl cellulose as a release controlling polymer.

In another embodiment, the present disclosure provides an extended release pharmaceutical composition comprising quetiapine, carboxymethyl ethyl cellulose (CMEC), and diluent selected from lactose, microcrystalline cellulose and dicalcium phosphate.

In another embodiment, pharmaceutical compositions of quetiapine comprise about 5% w/w to about 40% w/w carboxymethyl ethyl cellulose (CMEC) based on the total weight of the composition.

Another object of the present disclosure provides a method of preparing extended release compositions of quetiapine comprising the process of wet granulation or direct compression or dry granulation or extrusion and spheronization, wherein carboxymethyl ethyl cellulose is a release controlling polymer in the composition.

In an embodiment, pharmaceutically acceptable excipients of the core include one or more of diluents, disintegrants, binders, lubricants and glidants.

In another embodiment, the present disclosure provides a composition of a coating layer comprising one or more excipients selected from control release polymers, coating agents, opacifiers, taste masking agents, coloring agents, antitacking agents and the like.

In another aspect, a coating layer comprises one or more excipients selected from triethyl citrate, diacetylated monoglyceride, opadry yellow, isopropyl alcohol, glyceryl behenate and carboxymethyl ethyl cellulose.

An object of the present disclosure is to provide an extended release tablet composition comprising; a) a core containing quetiapine, carboxymethyl ethyl cellulose and one or more pharmaceutically acceptable excipients, and b) a coating layer comprising i) carboxymethyl ethyl cellulose and ii) one or more coating agents, wherein the carboxymethyl ethyl cellulose acts as non-gelling, hydrophobic release controlling polymer.

Another object of the present disclosure provides an extended release tablet composition comprising; a) a core containing quetiapine, carboxymethyl ethyl cellulose and one or more pharmaceutically acceptable excipients, and b) a coating layer comprising i) a non-gelling, hydrophobic release controlling polymer selected from carboxymethyl ethyl cellulose and glyceryl behenate and ii) one or more coating agents.

In a further aspect, the present disclosure provides an extended release pharmaceutical composition comprising carboxymethyl ethyl cellulose as a release controlling polymer which provides effective dissolution profiles of quetiapine for a period of up to about 20 hours.

### DETAILED DESCRIPTION

The term “quetiapine” as used herein includes quetiapine in the form of free base, in the form of a pharmaceutically acceptable salt thereof, amorphous quetiapine fumarate, quetiapine fumarate crystalline form or any isomer, derivative, hydrate, solvate, or prodrug or combinations thereof. In one embodiment, quetiapine is quetiapine fumarate.

The term “extended release” as used herein refers to a composition in which a drug is released over an extended period of time, such as over 4, 6, 10, 12, 15, 18, 20 or 24 hours, specifically over a period of up to 20 hours.

Accordingly, the present disclosure provides extended release compositions comprising quetiapine or its pharmaceutically acceptable salts, carboxymethyl ethyl cellulose (CMEC), a non-gelling, hydrophobic release controlling polymer, and one or more pharmaceutically acceptable excipients.

Carboxymethyl ethyl cellulose (CMEC) is a carboxymethyl and ethyl mixed ether of cellulose, comprising a white yellowish powder or grains without any odor and taste, almost insoluble in water and ethanol (95). Upon the addition of a methanol/dichloromethane mixture (1:1), a clear or slightly turbid viscous solution is formed which is insoluble in dilute sodium hydroxide testing solution. The carboxymethyl ethyl cellulose is a non-gelling, hydrophobic polymer.

As used herein a hydrophobic, non-gelling polymer is a polymer that is substantially insoluble in water and does not form a gel upon contact with water.

As used herein, the term core means an uncoated, compressed tablet. A core can be a substantially homogeneous mixture of drug and excipients, or inert core like non-pareil seeds or can be a plurality of particles such as spheroids containing the drug that are mixed with one or more excipients and compressed into a core. When the core is an inert core, the active pharmaceutical ingredient is coated onto the core such as by spraying. In general, a core contains quetiapine and in some embodiments carboxymethyl ethylcellulose.

A coating is a composition disposed on a core, generally substantially covering the core. The coating can be a functional or a non-functional coating, or multiple functional and/or non-functional coatings. A “functional coating” is a coating that modifies the release properties of the total formulation, for example, a sustained-release coating. A “non-functional coating” is a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition. In one embodiment, a coating is a substantially uniform coating.

Pharmaceutical compositions include extended release dosage forms such as tablets, capsules, caplets, granules, pellets, beads, pills, powders, sachets, particles, mini-tablets and the like.

Pharmaceutical compositions include extended release tablet compositions comprising quetiapine, carboxymethyl ethyl cellulose, a non-gelling, hydrophobic release controlling polymer and one or more pharmaceutically acceptable excipients.

Pharmaceutical compositions of quetiapine comprise about 5% w/w to about 40% w/w carboxymethyl ethyl cellulose, specifically about 10% w/w to about 30% w/w based on the total weight of the composition.

Pharmaceutical compositions comprise quetiapine and one or more excipients selected from the group consisting of release controlling polymers, diluents, disintegrants, binders, lubricants, glidants and plasticizers; characterized in that said composition comprise carboxymethyl ethyl cellulose as release controlling polymer.

Exemplary diluents include, lactose, sugar, starches, modified starches, mannitol, sorbitol, inorganic salts, cellulose derivatives (e.g., microcrystalline cellulose), calcium sulfate, xylitol, lactitol, starch, pregelatinized starch, kaolin, sucrose, mannitol, sorbitol, dextrates, dextrin, maltodextrin, dextrose, calcium carbonate, calcium sulfate, dibasic calcium phosphate, tribasic calcium phosphate, magnesium carbonate, magnesium oxide and the like and mixtures thereof.

The term “lubricant” as used herein means substances used in tablet formulations to reduce friction during tablet compression. Exemplary lubricants include calcium stearate, magnesium stearate, sodium stearyl fumarate, zinc stearate, talc, polyethylene glycols and the like and combinations thereof.

The term “glidant” as used herein means agents used in tablet and capsule formulations to improve flow-properties during tablet compression and to produce an anti-caking effect. Such compounds include, by way of example and without limitation, colloidal silica, calcium silicate, magnesium silicate, talc and the like and combinations thereof.

The term “binders” as used herein means substances used to provide adhesion of powder particles in tablet granulations. Exemplary binders include starches such as corn starch, potato starch, modified starches, pectin, microcrystalline cellulose, povidone, co-povidone, acacia, tragacanth, gelatin, liquid glucose, and pregelatinized starch, and the like and mixtures thereof.

The term “disintegrant” as used herein means a compound used in solid dosage forms to promote the disruption of the solid mass into smaller particles which are more readily dispersed or dissolved. Exemplary disintegrants include polacrillin potassium, croscarmellose sodium, crospovidone (e.g., KOLLIDON®, POLYPLASDONE®), polyvinylpyrrolidone, sodium starch glycolate (e.g., PRIMOGEL, EXPLOTAB®), carboxymethyl cellulose calcium, starches such as corn starch, potato starch, pre-gelatinized and modified starches, clays, bentonite, microcrystalline cellulose, and the like and combinations thereof.

The term “plasticizer” as used herein means a compound used in solid dosage forms to provide the desired plasticity to the coating. Exemplary plasticizers include propylene glycol, polyethylene glycol, triethyl citrate, acetyl triethyl citrate, acetyltributyl citrate, benzyl benzoate, chlorbutanol, dextrin, glycerin, glycerin monostearate, mannitol, lanolin alcohol, 2-pyrrolidine, sorbitol, triacetin, diacetylated monoglyceride, tri butyl citrate, triethanolamine, and the like and mixtures thereof.

The present disclosure also relates to process for preparing extended release pharmaceutical composition for oral administration, wherein the process includes admixing quetiapine, and at least one pharmaceutically acceptable excipient compatible with quetiapine.

A method of preparing extended release compositions of quetiapine comprises wet granulation or direct compression or dry granulation or extrusion and spheronization comprising carboxymethyl ethyl cellulose as a release controlling polymer.

A wet granulation process comprise the steps of (i) dry mixing quetiapine with one or more excipients, (ii) wet granulating the dry mix of step (i) using a binder solution to form granules followed by drying, (iii) lubricating the dried granules of step (ii), iv) followed by compression into tablets or alternatively the blend of step (iii) is filled in to capsules.

Direct compression process comprises the steps of: (i) dry mixing quetiapine with one or more excipients followed by blending, (ii) lubricating the blend obtained in step (i) by adding a lubricant, and (iii) finally compressing the blend of step (ii) into tablets.

Extrusion-spheronization process comprise the steps of: (i) blending quetiapine with one or more excipients to form a blended mixture, (ii) granulating the blended mixture of step (i) with binder solution to form a wet mass, (iii) extruding the wet mass of step (ii) followed by spheronization using a spherodizer to provide spheroids/spherical granules, and (iv) lubricating the spheroids/spherical granules with a lubricant and compressing in to tablets or filled in to capsules.

The tablets are optionally further coated by conventional coating techniques, well known to the person skilled in the art.

Accordingly, carboxymethyl ethyl cellulose and quetiapine are present in the core and/or in the coating of the tablet.

In one embodiment, a coating layer comprises one or more excipients selected from control release polymers, coating agents, opacifiers, taste masking agents, coloring agents, antitacking agents, and the like, and combinations thereof.

The coating layer comprises one or more of triethyl citrate, diacetylated monoglyceride, opadry, glyceryl behenate and carboxymethyl ethyl cellulose.

The present disclosure provides extended release tablet compositions comprising; a) a core containing quetiapine, carboxymethyl ethyl cellulose and one or more pharmaceutically acceptable excipients, and b) a coating layer comprising i) carboxymethyl ethyl cellulose and ii) one or more coating agents, wherein carboxymethyl ethyl cellulose acts as a non-gelling, hydrophobic release controlling polymer.

The present disclosure also relates to extended release tablet compositions comprising; a) a core containing quetiapine, carboxymethyl ethyl cellulose and one or more pharmaceutically acceptable excipients, and b) a coating layer comprising i) a non-gelling, hydrophobic release controlling polymer selected from carboxymethyl ethyl cellulose and glyceryl behenate and ii) one or more coating agents.

The glyceryl behenate used in quetiapine tablet coating is in an amount of about 0.5% w/w to about 3% w/w based on the total weight of the composition.

The following examples further illustrate the invention and do not limit the scope of the invention.

### Examples 1-6

### Extended Release Tablets of Quetiapine Prepared by Wet Granulation

| | | | | | | | |
|---|---|---|---|---|---|---|---|
| | | Ex. 1 | Ex. 2 | Ex. 3 | Ex. 4 | Ex. 5 | Ex. 6 |
| | | Mg/ | Mg/ | Mg/ | Mg/ | Mg/ | Mg/ |
| S. No | Ingredients | tablet | tablet | tablet | tablet | tablet | tablet |

I. Dry mix

| | | | | | | | |
|---|---|---|---|---|---|---|---|
| 1 | Quetiapine fumarate | 230 | 230 | 230 | 230 | 230 | 230 |
| 2 | Dicalcium phosphate | 50 | 110 | 68 | 85 | 110 | 105 |
| 3 | Lactose monohydrate | 65 | 60 | 40 | 50 | 50 | 50 |
| 4 | Microcrystalline | 100 | 0 | 0 | 0 | 0 | 0 |
| | cellulose | | | | | | |
| 5 | Carboxymethyl ethyl | 50 | 60 | 120 | 100 | 70 | 80 |
| | cellulose (CMEC) | | | | | | |

II. Granulation

| | | | | | | | |
|---|---|---|---|---|---|---|---|
| 6 | Isopropyl alcohol | q.s | q.s | q.s | q.s | q.s | q.s |
| 7 | Purified water | q.s | q.s | q.s | q.s | q.s | q.s |

III. Exra granulation

| | | | | | | | |
|---|---|---|---|---|---|---|---|
| 8 | Microcrystalline | 0 | 10 | 15 | 10 | 10 | 10 |
| | cellulose | | | | | | |
| 9 | Colloidal silicon | 0 | 5 | 2 | 0 | 0 | 0 |
| | dioxide | | | | | | |
| 10 | Magnesium stearate | 5 | 5 | 5 | 5 | 10 | 5 |
| | Weight of core tablet | 500 | 480 | 480 | 480 | 480 | 480 |

IV. Film coating

| | | | | | | | |
|---|---|---|---|---|---|---|---|
| 11 | Carboxymethyl ethyl | 0 | 4 | 0 | 2 | 4 | 5 |
| | cellulose (CMEC) | | | | | | |
| 12 | Triethyl citrate (TEC) | 0 | 2 | 0 | 1 | 2 | 0 |
| 13 | Diacetylated- | 0 | 0 | 0 | 1 | 2 | 2 |
| | monoglyceride | | | | | | |
| 14 | Opadry yellow* | 10 | 8.5 | 14.5 | 10.5 | 6.5 | 8 |
| 15 | Glyceryl behenate | 5 | 0 | 0 | 0 | 0 | 0 |
| 16 | Isopropyl alcohol | 0 | q.s | 0 | q.s | q.s | q.s |
| 17 | Purified water | q.s | q.s | q.s | q.s | q.s | q.s |
| | Weight of coated | 515 | 494.5 | 494.5 | 494.5 | 494.5 | 495 |
| | tablet | | | | | | |

*Composition of Opadry yellow: HPMC 6 cps, Titanium dioxide, PEG 400, Iron oxide yellow.

Brief manufacturing process:
i) Intra granular materials were sifted through #30 mesh, and blended together,
ii) the blended material of step no (i) was loaded in a rapid mixer granulator and granulated using granulating solution for 15 minutes,
iii) the granules of step no (ii) were dried until loss on drying is 2.0-3.0% w/w,
iv) dried granules of step (iii) were milled and passed through #20 mesh,
v) extra granular materials were sifted together through #20 mesh,
vi) materials of step (iv) and (v) were blended together and compressed into tablets,
vii) the core tablets obtained in step (vi) were further film coated using coating solution.

### Preparation of Coating Solution:

i) carboxymethyl ethyl cellulose/glyceryl behenate were dissolved in isopropyl alcohol to provide a solution,
ii) remaining coating ingredients were dissolved in purified water to provide a solution,
iii) solutions obtained in step (i) and (ii) were mixed under stirring to form a uniform coating solution.

### Example 7

### Extended Release Tablets of Quetiapine Prepared by Direct Compression

| | | |
|---|---|---|
| S. No | Ingredients | Mg/tablet |

I. Dry mix

| | | |
|---|---|---|
| 1 | Quetiapine fumarate | 230 |
| 2 | Dicalcium phosphate | 65 |
| 3 | Lactose monohydrate | 50 |
| 4 | Carboxymethyl ethyl cellulose (CMEC) | 120 |

II. Lubrication

| | | |
|---|---|---|
| 5 | Microcrystalline cellulose | 10 |
| 6 | Magnesium stearate | 5 |
| | Weight of core tablet | 480 |

IV. Film coating

| | | |
|---|---|---|
| 7 | Carboxymethyl ethyl cellulose (CMEC) | 4 |
| 8 | Diacetylatedmonoglyceride | 2 |
| 9 | Opadry yellow* | 9 |
| 10 | Isopropyl alcohol | q.s |
| 11 | Purified water | q.s |
| | Weight of coated tablet | 495 |

*Composition of Opadry yellow: HPMC 6 cps, Titanium dioxide, PEG 400, Iron oxide yellow.

### Brief Manufacturing Process:

i) Intra granular materials were sifted through #30 mesh, and blended together,
ii) extra granular materials were sifted together through #30 mesh,
iii) materials of step (i) and (ii) were blended together and compressed into tablets,
iv) the core tablets obtained in step (iii) were further film coated.

The tablets prepared from examples 1 to 7 were tested for their dissolution profile initially for two hours in USP Type I1 Apparatus (Paddle) at 100 rpm, using 750 ml 0.1N HCl and followed by twenty hours in USP Type I1 Apparatus (Paddle) at 100 rpm, using 1000 ml phosphate buffer having pH 6.2. The results obtained were compared with the marketed formulation (Seroquel XR®).

| | Percentage of drug release | | | | | | | |
|---|---|---|---|---|---|---|---|---|
| Time | Seroquel | | | | | | | |
| (hr) | XR ® 200 mg | Ex. 1 | Ex. 2 | Ex. 3 | Ex. 4 | Ex. 5 | Ex. 6 | Ex. 7 |

0.1N HCl, 750 ml, 100 rpm, USP Type-II Apparatus

| | | | | | | | | |
|---|---|---|---|---|---|---|---|---|
| 1 | 22 | 66 | 18 | 39 | 30 | 22 | 18 | 28 |
| 2 | 39 | 92 | 36 | 51 | 45 | 40 | 34 | 48 |

pH 6.2 Phosphate buffer, 1000 ml, 100 rpm, USP Type-II Apparatus

| | | | | | | | | |
|---|---|---|---|---|---|---|---|---|
| 4 | 44 | 95 | 50 | 55 | 49 | 48 | 41 | 50 |
| 6 | 55 | 93 | 65 | 59 | 58 | 63 | 48 | 70 |
| 8 | 66 | 94 | 78 | 64 | 65 | 79 | 55 | 76 |
| 10 | 74 | 96 | 89 | 69 | 72 | 88 | 64 | 86 |
| 12 | 84 | 93 | 92 | 72 | 80 | 92 | 76 | 93 |
| 14 | 86 | 95 | 95 | 76 | 88 | 96 | 83 | 96 |
| 16 | 88 | 91 | 97 | 80 | 91 | 98 | 91 | 99 |
| 20 | 94 | 95 | 100 | 85 | 94 | 100 | 95 | 101 |

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

We claim:

1. An extended release composition comprising; i) quetiapine or a pharmaceutically acceptable salt, polymorph, solvate, or hydrate thereof, ii) carboxymethyl ethyl cellulose (CMEC), a non-gelling, hydrophobic release controlling polymer and iii) one or more pharmaceutically acceptable excipients.

2. The composition of claim 1, wherein the quetiapine is quetiapine fumarate.

3. The composition of claim 1, wherein the composition comprises quetiapine fumarate and carboxymethyl ethyl cellulose, present in a core or in a coating or in both.

4. The extended release composition of claim 1, wherein the pharmaceutically acceptable excipients comprises one or more of diluents, binders, disintegrants, lubricants, glidants and plasticizers.

5. The extended-release composition of claim 1, comprising;
a) a core comprising quetiapine, carboxymethyl ethyl cellulose, and one or more pharmaceutically acceptable excipients, and
b) coating layer disposed on the core comprising i) carboxymethyl ethyl cellulose and ii) one or more coating agents.

6. An extended release tablet composition comprising; a) a core comprising quetiapine, carboxymethyl ethyl cellulose and one or more pharmaceutically acceptable excipients, and b) a coating layer disposed on the core comprising i) carboxymethyl ethyl cellulose (CMEC), a non-gelling, hydrophobic release controlling polymer and ii) one or more coating agents.

7. The extended release tablet composition of claim 6, wherein the core is produced by wet granulation, dry granulation, or extrusion and spheronization.

8. The extended release composition of claim 1 or 6, wherein the composition comprises about 5% w/w to about 40% w/w of carboxymethyl ethyl cellulose based on total weight of the composition.

9. The extended release composition of claim 1, in the form of an oral solid dosage form selected from a tablet, a caplet, pellets, a capsule, granules, a pill, powder, or a sachet.

10. A method of making a quetiapine formulation by wet granulation comprising (i) dry mixing quetiapine with carboxymethyl ethyl cellulose (CMEC), (ii) wet granulating the dry mix of step (i) using a binder solution to form granules followed by drying, (iii) lubricating the dried granules of step (ii) with a lubricant, iv) followed by compression of the lubricated granules into tablets.

11. The method of claim 10, further comprising coating the tablets.

12. The method of claim 11, wherein the tablet coating comprises carboxymethyl ethyl cellulose (CMEC).

13. The pharmaceutical composition comprising therapeutically effective amount of quetiapine according to any of the preceding claims is useful in treating schizophrenia.
